(19) United States
(12) Patent Application Publication
(10) Pub. No.: US 2005/0279579 A1
(43) Pub. Date: Dec. 22, 2005

(54) Retail display product combination and methods

(76) Inventors: Jeremy I. Milk, Oakland, CA (US); Jennifer M. Miller, Oakland, CA (US); Glenn R. Savage, Oakland, CA (US)

Correspondence Address: THE CLOROX COMPANY, 1221 BROADWAY PO BOX 2351, OAKLAND, CA, 94623, US

(21) Appl. No.: 10/873,919

(22) Filed: Jun. 22, 2004

(51) Int. Cl. E04H 3/00; A61F 13/15

(52) U.S. Cl. 186/52

(57) ABSTRACT

A display and a method of displaying articles for sale in a mass retail environment. The method comprises displaying multiple product containers in an adjacent fashion, in a row, on a shelf, where each container includes the same brand display. The method further comprises including product containers having a common characteristic selected from the group consisting of fragrance, product color, container shape, container color and combinations thereof. The products include at least two product types selected from the group consisting of personal care products, home care products, and laundry care products.

### BACKGROUND OF THE INVENTION

1. Field of the Invention

This invention relates to shelving and displays for marketing a combination of home care, laundry and personal care products. More specifically, the invention comprises using a particular retail shelf display arrangement and method to promote the sale of traditionally unrelated products in a retail environment.

2. Description of the Related Art

Mass retail store shelves are typically arranged and configured to optimize exposure of products to the customer without serious regard for marketing. More recently, mass retailers have begun to permit manufacturers to provide marketing support at the retail shelf, by placing a display card or some other attractive device adjacent the manufacturer's product. Other examples are coupon dispensers positioned near products, advertisements located in shopping carts and advertisements on floor displays.

Even though there are many retailers that sell groups of products from a single manufacturer in a single location within a store (e.g. a “Sony Store” within an electronics retail store, or a “Levi's Store” within an apparel store), it has long been a philosophy of mass retail shelving that the same products displaying different manufacturers' brand names are shelved together. A large number of such products can then be viewed quickly, permitting the purchaser to compare them for quality and value, and to easily find all offerings of specific products in one place. This is particularly true for mass retailers such as large grocery chains, drug stores, food stores and club stores that sell consumer goods (as opposed to specialty home stores and boutiques). Thus, for example, one finds a “household cleaning products aisle” in the store displaying many brands of different manufacturers' cleaning products. In a separate location in the store, the purchaser might find a “paper products” aisle or an “automotive products” aisle, and so on.

Manufacturers of consumer goods often produce multiple products under the same brand that are sold at retail and which are displayed in these different sections or aisles in a store. Mass retailers have resisted the shelving of these products together by brand as this violates the basic philosophy of permitting the customer to comparison shop across brands, and can make it more difficult for customers to locate similar products within the store. Nevertheless, some retailers have permitted such “brand blocks” on certain occasions where a single brand might be having a promotion. “Brand blocks” are also more likely where the products are considered related—for example cleaning brushes for the bathroom, and mops for floor care throughout the home, or in boutique or home specialty stores. “Brand blocks” are also used in the personal care area of some retailers, for example with cosmetics.

### SUMMARY OF THE INVENTION

The invention comprises a method of displaying articles for sale in a mass retail environment, which method comprises displaying multiple product containers in an adjacent fashion, in a row, on a shelf, where each container includes the same brand display. The method according to the invention further comprises including product containers having a common characteristic selected from the group consisting of fragrance, product color, container shape, container color and combinations thereof. Importantly the method further comprises the step of displaying, in the combination of articles for retail sale, a combination of products consisting of at least two product types selected from the group consisting of personal care products, home care products, and laundry care products.

The invention further comprises a display for the sale of retail products comprising displaying multiple product containers in an adjacent fashion, in a row, on a shelf in a mass retail store, where each container includes the same brand display. The display according to the invention further comprises including product containers having a common characteristic selected from the group consisting of fragrance, product color, container shape, container color and combinations thereof. Importantly the display further comprises the step of displaying, in the combination of articles for retail sale, a combination of products consisting of at least two product types selected from the group consisting of personal care products, home care products, and laundry care products.

### DESCRIPTION OF THE FIGURES

The invention will be best understood by reference to the appended drawings in which:

FIG. 1 is a display shelf including a horizontal array of a single brand of products forming a brand-block and having additional related features; and

FIG. 2 is a display shelf including a horizontal array of products comprising a first set of products having related characteristics and second set of the same products having related characteristics, all of the products of a single brand.

### DETAILED DESCRIPTION OF THE INVENTION

The invention provides a novel manner in which to shelve and display a selected set of products of a single brand and including products that have traditionally not been displayed or shelved together, in a mass-market retail environment. This adjacent shelving “brand-block” provides the manufacturer/supplier with leveraged marketing between brands and can improve profits for both the manufacturer and the retailer. According to the invention, “mass” retail includes large drug, food, club and grocery stores and is intended to exclude specialty and boutique stores. Examples of mass retailers include, for example, Walmart, K-Mart and Target stores.

In one aspect of the invention the product array comprises a display in which at least two different products can be placed on a mass retail shelf, the products being selected from the group of home cleaning, personal care and laundry care. Typically, however, such a display will include more than two different products and will include at least one each of a home cleaning, personal care and laundry care product.

Though this mix of product types has not previously been sold as a brand-block in a mass retail environment, an important part further part of the invention relates to combining the products in a manner that displays and emphasizes contrived and attractive like attributes. By using these attributes the new brand block presents a cohesive and attractive set of products in the display, persuading the customer to purchase the range of products rather than an individual one.

Thus, each of the home cleaning, personal care and laundry care products will display the same brand name in some form, prominently displayed on at least a portion of the container, preferably in the form of a stylized logo or trademark illustration. As necessary elements along with the branding, however, the additional contrived attributes will also be used. These contrived attributes the products may share include, but are not limited to, color (whether of the label, the product itself or the container), lack of color (transparency), scent, shape and size. It is preferred that at least color and scent are consistent between the product containers.

In another preferred embodiment, each of the containers for the products is transparent and the products within the containers are the same color or shades of the same color, and that the color or shades of color are matched to a scent which is also consistent throughout the products in the display.

In another aspect of the invention, in order to present greater consistency with respect to the top height of the containers on the display, one or more of the group of products forming the brand block will be displayed on a support or “podium.” In a preferred form of this aspect of the invention, the podium will be used to support a “shorter” product container (or containers) that is (are) displayed alongside “taller” containers. We have found that the use of such a podium in this manner presents a more attractive and cohesive display.

Where a podium is used, the face of the podium may be used to display a logo, trademark or text that further enhances the customer-facing display. The podium can be multi-tiered.

In another aspect of the invention, the display may comprise a shelving unit. It may be advantageous for the manufacturer of the retail products to include such a shelving unit for use at retail, since a custom shelving unit may create a preferred setting for the products. In a preferred form, the shelving unit will be sized to fit comfortably within the specific confines of standard retail shelving and will be shaped and colored to emphasize the cohesive nature of the combination of products that are placed in the shelf as discussed herein.

Within the meaning of this invention, “home cleaning” or “home care” products means products that are used to clean, sanitize and/or provide a scent to areas of the home and which are not generally applied to the body or hair or used in the laundry. Home cleaning or home care products include dish soap, all purpose cleaners, glass cleaner, surface cleaner, surface polisher (eg: steel, wood etc), tub and tile cream cleaner, dish purifying wash, automatic dishwashing powder, automatic dishwashing liquid, dilutable cleaners, vacuum fragrance (powders or beads), surface scrubs, cream cleanser, abrasives, scented candles, fragrance wands, fragrance plug-ins, fragrance diffuser, room spray/mist, fabric deodorizer, wipes (for wood, glass, counter, kitchen, and/or all surfaces), floor cleaner, wood cleaner, pot pourri, carpet cleaner and refills and combinations thereof.

“Personal care” products means products that are applied to the hair and/or the body, directly or indirectly. Examples of personal care products include hand soap and hand lotion, hand scrub, bar soap, body lotion, bubble bath, massage oil, body scrub, eau de toilette, body spray, shower gel, shampoo, conditioner, hair care and styling products, deodorant, body cream, sun lotion, body mist, bath salts, body oil, body mask, face mask, cleansing milk, toner, lip balm, foot treatments (cream, oil or mist), shaving cream, after-shave lotion, talcum powder, lip or body treatments or balm and refills and combinations thereof.

“Laundry care” products means, as part of this invention, products that are applied to clothing or other items cleaned in the laundry (such as bedding), either in the washer or dryer or directly applied before or separately from the washer or dryer. These products will clean, impart a scent or both. Examples of laundry care products include fabric softener, sheet spray, clothing spray, laundry fragrance, dryer sachets, perfumed sachet, dryer sheets, laundry soap, laundry detergent, detergent for delicates, ironing spray, starch, fragrance sheets, drawer liner sheets, cedar closet spray, linen water and refills and combinations thereof.

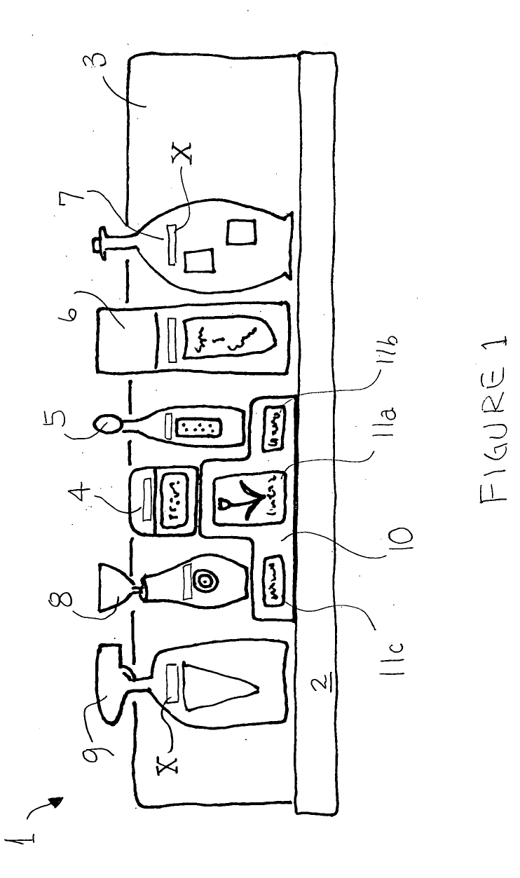

An example of a display arrangement according to the invention is shown in FIG. 1. As shown therein, a display 1 according to the invention will, in this form, include a series of product containers 4-9 positioned adjacent one another. While products 4-9 can be positioned on the retail shelf as shown, in this embodiment, the display includes a provided shelving unit comprising a supporting base 2 and back 3. The shelving unit can be made of any appropriate material and decorated as appropriate for creating the most attractive setting for the products.

This embodiment includes optional podium or supporting stand 10, in this case having a multi-tiered structure such that products 4, 5 and 8 can be positioned at roughly the same “height” as taller containers 6, 7 and 9. The podium may include information for the consumer, for example labels 11a, 11b and 11c, including key information about the related characteristics of the products (such as scent and color) and optionally the brand that also appears on each of the container labels.

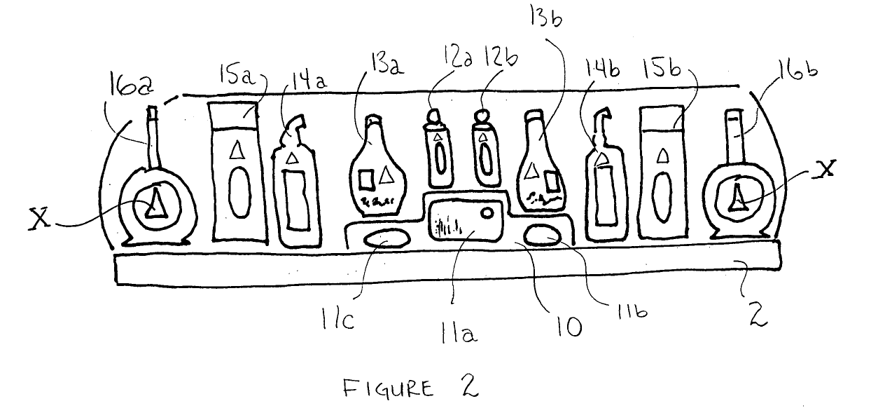

Each of product containers 4-9 includes a label, facing the customer, which will include the same brand (labeled “X” in each of FIGS. 1 and 2), typically a logo or other stylized trademark. Additionally the label or labels on each will include product information, and in a preferred form of the invention, the (same) scent for the product in each container.

In this embodiment the display includes three types of products that have not previously been included in a brand block display and with the common characteristics included here, such as product or container color (the same color or shades of the same color) and scent. In FIG. 1, product 9 represents an “all purpose cleaner”, which is a “home care” or “home cleaning” category product. On the other hand, product container 4 represents a personal care product such as hand lotion, and product container 7 represents a laundry care product such as a fabric softener.

While a key aspect of the invention is to include at least two of the three types of products (home care, personal care and laundry care), the display will advantageously include more than one type of product from each of these categories as well. In FIG. 1, for example, container 8 represents another personal care product (for example hand soap), container 6 represents a second laundry care product (for example sheet and clothing spray), and container 5 represents a second home care product (for example dish soap).

FIG. 2 shops another preferred example according to the invention. The display arrangement of Figure is also a brand block, but in this case includes two “families” of products displayed as a brand block. Thus, product containers 12a-16a are a first related set of products and containers 12b-16b form a second set of products. Each set of the products has the key characteristics of the invention: a brand block including at least two products selected from the group consisting of home care, laundry care and personal care. Additionally, in this embodiment, two brand blocks according to the invention are placed side by side. Since all of the containers bear a single brand, there is cohesiveness throughout the display. However, the display is made more attractive by having products 12a-16a of a first scent and first color or shades of a single color along with products 12b-16b having a different second scent and a different second color or shades of a single color.

As in FIG. 1, FIG. 2 includes a podium for creating interest for the consumer's eye and as a vehicle for permitting text or graphics to appear on the face of the display on labels 11a-11c.

By combining the brand block approach with a single color and scent combination (as in FIG. 1) or with a dual color and scent combination (as shown in FIG. 2) the display according to the invention becomes more attractive and opens the possibility of changing retailers' approaches. While this detailed description includes specific examples according to the invention, those skilled in the art will appreciate that there are many variations of these examples that would nevertheless fall within the general scope of the invention and for which protection is sought in the appended claims.

1-3. (canceled)

4. A mass retail display combination comprising a set of product containers, each one of the set positioned adjacent at least one other of said set, each of said containers including a label comprising a same brand insignia, wherein one or more of the product containers are formed of transparent materials and the product within the transparent containers is a shade or the same color as other containers or products in the display, and wherein the products contained within the product containers include at least two members selected from the group consisting of home cleaning products, laundry care products and personal care products.

5. The display according to claim 4 wherein each of the products in the display comprises the same scent.

6. The display according to claim 4 wherein at least major portions of the product containers are shades of the same color, each of the containers is transparent, each of the products in the display comprises the same scent, and the same scent name appears on each label.

7. (canceled)

8. The display according to claim 4 wherein the display comprises at least four containers and at least two of the containers have the same shape.

9. The display according to claim 4 wherein at least one of the containers on the display is supported on a podium.

10. (canceled)

11. A mass retail display combination comprising a first set of product containers, each one of the set positioned adjacent at least one other of said set, each of said containers including a label comprising a same brand insignia, the products contained within the product containers including at least one product each selected from the group consisting of home cleaning products, laundry care products and personal care products.

12. The display according to claim 11 wherein at least major portions of the product containers are a same first color or shades of a same first color.

13. The display according to claim 11 wherein each of the containers is transparent and each of the products in the containers is a same first color or shades of a same first color.

14. The display according to claim 11 wherein each of the products in the display comprises a same scent, and a same scent name appears on each label.

15. The display according to claim 11 further comprising a second set of product containers, each one of the second set positioned adjacent at least one other of said second set, each of said second set of containers including a label comprising the same brand insignia as the first set of containers, the products contained within the second set of product containers including at least one product each selected from the group consisting of home cleaning products, laundry care products and personal care products and wherein each of the second set of products in the display comprises a second scent different than the first scent and the second scent name appears on each second container set label.

16. The display according to claim 11 wherein at least one of the first containers on the display is supported on a podium.

17. The display according to claim 15 wherein at least one of the first containers on the display is supported on a podium and wherein at least one of the second set of containers is supported on the same podium.

18. A method for displaying products for retail sale comprising the steps of
a. placing on a shelf in a mass retail store a first product in a labeled container displaying a brand, the product comprising a laundry care product having a first fragrance; and
b. placing a labeled, second product container on the shelf, the second also displaying the brand, and adjacent the first product, the second product comprising a product selected from either a personal care product or a home cleaning product and including said first fragrance.
